(19) United States
(12) Patent Application Publication
(10) Pub. No.: US 2025/0061979 A1
(43) Pub. Date: Feb. 20, 2025

(54) PREDICTING MOLECULE PROPERTIES USING GRAPH NEURAL NETWORK

(71) Applicant: Kebotix Inc., Cambridge, MA (US)

(72) Inventors: Dennis Sheberla, Bedford, MA (US); Kevin Ryan, Watertown, MA (US)

(73) Assignee: Kebotix Inc., Cambridge, MA (US)

(21) Appl. No.: 18/722,059

(22) PCT Filed: Dec. 22, 2022
(86) PCT No.: PCT/US2022/053801

§ 371 Date: Jun. 20, 2024

Related U.S. Application Data
(60) Provisional application No. 63/293,608, filed on Dec. 23, 2021.

(51) Int. Cl. G16C 20/70; G06N 3/0464; G06N 3/048; G16C 20/30

(52) U.S. Cl. CPC G16C 20/70; G06N 3/0464; G06N 3/048; G16C 20/30

(57) ABSTRACT

The techniques described herein relate to computerized methods and apparatuses for predicting properties of molecules using a neural network. The neural network may include one or more layers to convert atom and bond features of an input molecule into respective atom and bond representations; a graph neural network configured to update the atom and bond representations; a molecule layer configured to convert the updated atom and bond representations into a molecule representation; and a target layer configured to predict one or more properties of the molecule based on the molecule representation. Prediction may include a regression operation to predict a single property value of the molecule, or a classification operation to predict probabilities of the molecule belonging to respective classes of a plurality of classes. The graph neural network may include a graph transformer network. The graph neural network may include a graph convolutional neural network.

### CROSS-REFERENCE TO RELATED APPLICATIONS

This application claims the benefit of U.S. Provisional Application No. 63/293,608, filed Dec. 23, 2021, entitled, “PREDICTING MOLECULE PROPERTIES USING GRAPH TRANSFORMER NEURAL NETWORK,” the entire content of which is incorporated herein by reference.

### BACKGROUND

For chemical processes and analyses, it can be desirable to understand information about molecule(s) of interest. For example, it can be desirable to understand various properties of the input molecule(s) to a chemical process.

### SUMMARY

Some embodiments are directed to a system for predicting properties of a molecule, the system comprising: at least one processor configured to provide an input molecule to a neural network model and use the neural network model to predict one or more properties of the input molecule. The neural network may include: an atom embedding layer configured to convert atom features of the input molecule to an atom representation; a bond embedding layer configured to convert bond features of the input molecule to a bond representation; a graph neural network comprising at least one layer configured to update the atom representation base at least in part on the bond representation; a molecule embedding layer configured to generate a molecule representation based on the updated atom representation; and a target layer configured to predict one or more properties of the molecule based on the molecule representation. In some examples, the graph neural network may be a graph transformer network. In some examples, the graph neural network may be a graph convolutional neural network (GCNN).

Some embodiments are directed to method for predicting properties of a molecule, the method comprising, using at least one processor, using a neural network model to predict one or more properties of an input molecule. Using the neural network to predict one or more properties may include: converting atom features of the input molecule to an atom representation; converting bond features of the input molecule to a bond representation; using a graph neural network comprising at least one layer to update the atom representation base at least in part on the bond representation; generating a molecule representation based on the updated atom representation; and predicting one or more properties of the molecule based on the molecule representation. In some examples, the graph neural network may be a graph transformer network. In some examples, the graph neural network may be a GCNN.

Some embodiments are directed to a non-transitory computer-readable media comprising instructions that, when executed, cause at least one processor to perform operations comprising: using a neural network model to predict one or more properties of an input molecule. Using the neural network model to predict one or more properties include: converting atom features of the input molecule to an atom representation; converting bond features of the input molecule to a bond representation; using a graph neural network comprising at least one layer to update the atom representation base at least in part on the bond representation; generating a molecule representation based on the updated atom representation; and predicting one or more properties of the molecule based on the molecule representation. In some examples, the graph neural network may be a graph transformer network. In some examples, the graph neural network may be a GCNN.

### BRIEF DESCRIPTION OF THE DRAWINGS

Additional embodiments of the disclosure, as well as features and advantages thereof, will become more apparent by reference to the description herein taken in conjunction with the accompanying drawings. The components in the figures are not necessarily to scale. Moreover, in the figures, like-referenced numerals designate corresponding parts throughout the different views.

FIG. 1A illustrates an example neural network for predicting properties of molecules, according to some embodiments.

FIG. 1B illustrates example components of a target layer of a neural network for performing regression operation in predicting properties of molecules, according to some embodiments.

FIG. 1C illustrates example components of a target layer of a neural network for performing classification operation in predicting properties of molecules, according to some embodiments.

FIGS. 1D-1E illustrate example components of additional layers in a neural network as shown in FIG. 1A, according to some embodiments.

FIG. 2 illustrates an example graph transformer layer of a neural network for performing regression operation in predicting properties of molecules, according to some embodiments.

FIG. 3 illustrates an example graph transformer layer of a neural network for performing classification operation in predicting properties of molecules, according to some embodiments.

FIGS. 4A-4C illustrate portions of an implementation of a neural network for performing regression operation in predicting properties of molecules, according to some embodiments.

FIGS. 5A-5C illustrate portions of an implementation of a neural network for performing classification operation in predicting properties of molecules, according to some embodiments.

FIG. 6 illustrates an example GCNN layer of a neural network for predicting properties of molecules, according to some embodiments.

FIG. 7A illustrates an example of index-select operation, according to some embodiments.

FIG. 7B illustrates an example of sparse matrix for representing atom connectivities, according to some embodiments.

FIG. 8 shows an illustrative implementation of a computer system that may be used to perform any of the aspects of the techniques and embodiments disclosed herein, according to some embodiments.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the invention is thereby intended.

A neural network system for use in a chemical process may be trained to use information about an input molecule to predict one or more properties of the input molecule. The molecule information may include, for example, information about one or more atoms in the molecule, bonding relationship(s) among bonded atom pairs, etc. Thus, the neural network system may need to learn the atom features, bond features, etc. of various molecules in training dataset(s). The inventors have appreciated that, considering the complexity of such molecule information, including atom features and bond features of molecules (e.g., which can be quite complex for large molecules), conventional neural networks may not be adequate to predict the properties of molecules efficiently. For example, large numbers of layers (e.g., as many as 80-100 layers) of a conventional neural network may be needed. Additionally, or alternatively, such networks may suffer from a lack of sufficient training data. For example, a molecule of interest for property prediction may not have a matching structure to that of the training data. As a result, conventional networks may not be able to sufficiently predict molecule properties.

Accordingly, the inventors have developed techniques for predicting properties of molecules using a neural network architecture that leverages a graph neural network. Described herein are various techniques, including systems, computerized methods, and non-transitory instructions, that configure, train, and/or run a neural network for predicting properties of molecules. The neural network may include one or more layers configured to convert one or more atom features and bond features of an input molecule into atom and bond representations; a graph neural network configured to update the atom and bond representations; a molecule layer configured to convert the updated atom and bond representations into a molecule representation; and a target layer configured to predict one or more properties of the molecule based on the molecule representation. In some embodiments, the graph neural network may be a graph transformer network. In some embodiments, the graph neural network may be a GCNN. It is appreciated that the graph neural network may be any other suitable neural network.

In some embodiments, the input to the neural network can be a representation of a molecule of interest. For example, the representation can be a graph that includes a set of atoms in a specific order that provides information about which pairs of atoms are related to each other via bonds. The neural network can be configured to predict one or more properties of the input molecule, such as oxidation, melting point, flash point, etc. While some conventional approaches attempt to represent molecules as a fixed number of dimensions, the inventors have appreciated that such an approach can be limiting since it can be undesirable to have a fixed data size (e.g., since molecules can have variable numbers of atoms and bonds). Accordingly, the inventors have developed techniques for developing a fingerprint for each molecule that is used to predict one or more properties of the molecule. The techniques include generating an atom embedding and bond embedding for the input molecule, and leveraging a graph neural network to predict the properties of the input molecule (e.g., via regression and/or classification) based on the generated atom embedding and bond embedding.

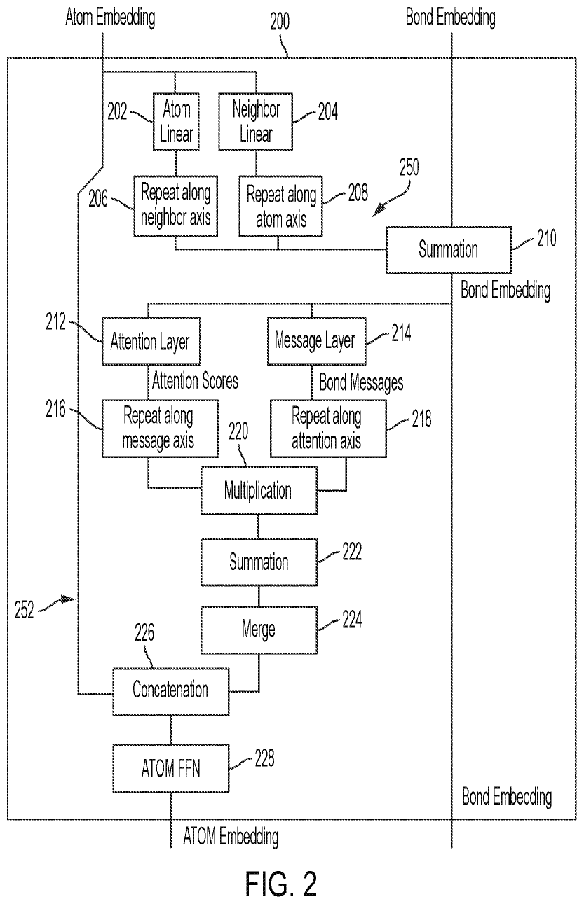

In some embodiments, the system may use various embedding techniques to generate the atom and bond representations. For example, one or more atom and/or bond features may be represented in one-hot representation, and in certain optimal dimensions. In some non-limiting embodiments, the graph neural network may be a graph transformer network. The graph transformer network may include multiple graph transformer layers. The graph transformer network may include an attention network to update the atom and/or bond representations to allow the neural network to focus on certain features, such as bond features that represent relationships of atoms that are bonded (e.g., as opposed to atoms that are not bonded). Such configuration allows the neural network to converge faster and run more efficiently and more accurately (e.g., since the attention network can filter out information that is irrelevant for the property prediction(s)). The inventors have further appreciated that use of one or more residual connections can improve the prediction (e.g., regression/classification). In some embodiments, a residual connection is used to update the bond embedding with the atom embedding, which can improve the attention network as discussed further herein (e.g., in conjunction with FIG. 2).

Whereas various embodiments have been described, it will be apparent to those of ordinary skill in the art that many more embodiments and implementations are possible. Accordingly, the embodiments described herein are examples, not the only possible embodiments and implementations. Furthermore, the advantages described above are not necessarily the only advantages, and it is not necessarily expected that all of the described advantages will be achieved with every embodiment.

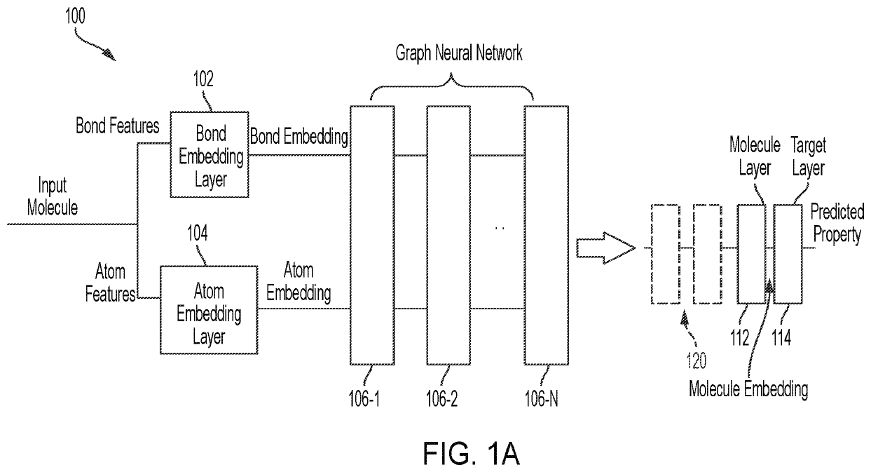

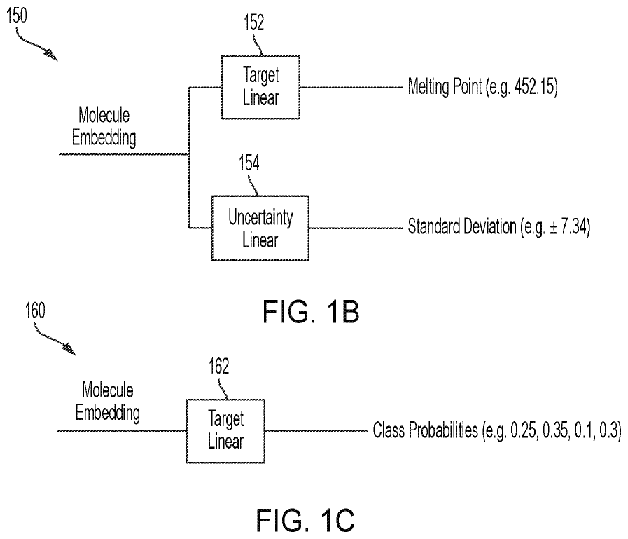

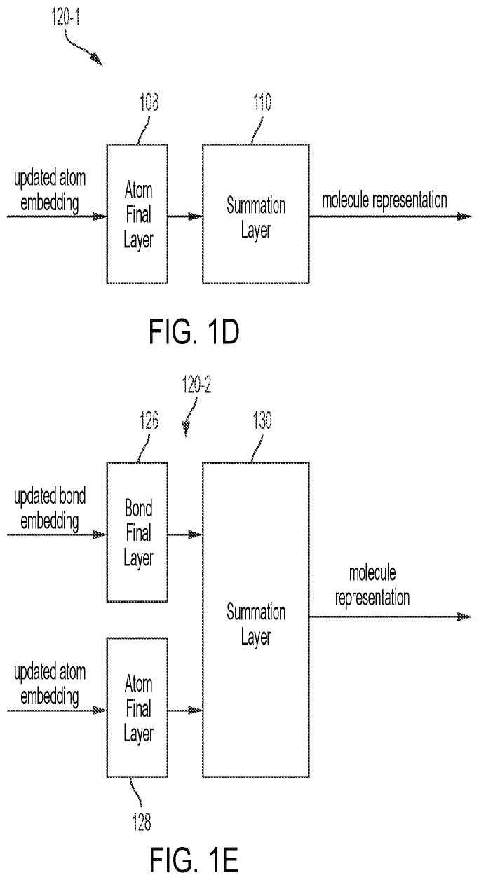

FIG. 1A illustrates an example neural network 100 in a system for predicting properties of molecules, according to some embodiments. The system may include at least one processor configured to provide an input molecule to a neural network model and use the neural network model to predict one or more properties of the input molecule. In some embodiments, neural network 100 may include various layers and operations. A layer (e.g., represented by a rectangular) may include trainable parameters (e.g., weights) that may be trained and used for prediction. A layer may propagate information along the feature axis. For example, a layer may be operated to extract certain features. A neural network layer may perform a transformation from one feature dimensionality to another. This choice of dimensionality is made when the network is configured at the start of training and prediction, and it remains constant for all possible inputs. An operation (e.g., represented by a rounded rectangular), on the other hand, does not contain trainable parameters. An operation may propagate information along other axes and may change the dimensionality of the data. For example, an operation may include summation, concatenation, multiplication etc.

In some embodiments, with reference to FIG. 1A, neural network model 100 may include: an atom embedding layer 104 configured to convert atom features of the input molecule to an atom representation (e.g., atom embedding); a bond embedding layer 102 configured to convert bond features of the input molecule to a bond representation (e.g., bond embedding); a graph neural network 106 comprising at least one layer (e.g., 106-1, 106-2, . . . 106-N), where the at least one layer may be configured to update the atom representation based at least in part on the bond representation; a molecule embedding layer 112 configured to generate a molecule representation based on the updated atom representation; and a target layer 114 configured to predict one or more properties of the molecule based on the molecule representation. Additionally and/or alternatively, neural network 100 may include one or more additional layers/operations (e.g., 120), the details of which will be further described.

With further reference to FIG. 1A, the input molecule to the neural network 100 may be represented in any suitable representation, such as SMILES. A molecule representation may include information about the one or more atoms in the molecule and information about one or more pairs of atoms, in which the atoms in each pair are bonded. Information about the atoms and bonding relationships of atoms in a molecule may be referred to atom features and bond features, respectively. For example, in a non-limiting example, the atom features of the input molecule may include the number of atoms in the input molecule, and, for each atom in the input molecule, an atom number, a chirality, a formal charge, and/or a hydrogen count.

These atom features may be represented in any suitable data representation. In a non-limiting example, the atom number may be represented in a one-hot representation to enable fast process. For example, the atom number may be a 118-dimensional vector capable of representing up to 118 atoms. It is appreciated that other dimensions may also be possible. Similarly, chirality may be represented in a one-hot representation, such as a 4-dimensional vector. It is appreciated that other dimensions may also be possible. In some examples, the formal charge and the hydrogen count may each be a scalar (e.g., a single value). In some examples, the features described above may be concatenated to form a vector, e.g., a 124-dimensional vector in the example above. Other representations may also be possible.

In some embodiments, the bond features of an input molecule may include, for each bond relationship, a bond type, a bond direction (orientation), and/or a shortest path distance. In some examples, the bond type may be represented in a one-hot representation, e.g., in 22-dimension (or other suitable dimensions). The bond direction may be represented in a one-hot representation, e.g., in 7-dimension (or other suitable dimensions). The shortest path distance, which is provided as input features to the graph neural network, may be represented in positional encoding. The dimension of the positional encoding may be selected so that the dimension may be a suitable value: it is not too low to cause the network to have difficult to understand the positional encoding; and the dimension is not too high to cause substantial calculation overhead and/or drowning out the other bond features in the system. In some examples, the positional encoding may be represented in 16-dimension. It is appreciated that other dimensions may also be possible. In the example above, the bond features may be concatenated to form a vector, e.g., a 45-dimensional vector in the example above. Other representations may also be possible.

In some embodiments, the shortest path distance may be calculated using the molecule's adjacency matrix and a shortest path algorithm. Examples of shortest path algorithms include Floyd-Warshall algorithm (as described in Floyd, Robert W., “Algorithm 97: Shortest Path,” Communications of the ACM. 5 (6): 345, June 1962), Dijkstra's algorithm with Fibonacci heaps (as described in Fredman, Michael Lawrence; Tarjan, Robert E., “Fibonacci heaps and their uses in improved network optimization algorithms,” Journal of the Association for Computing Machinery. 34 (3): 596-615, July 1987), Bellman-Ford algorithm (as described in Bellman, Richard, “On a routing problem,” Quarterly of Applied Mathematics. 16: 87-90, 1958), and Ford, Lester R. Jr., Network Flow Theory. Paper P-923. Santa Monica, California: RAND Corporation, Aug. 14, 1956), and/or Johnson's algorithm (as described in Johnson, Donald B., “Efficient algorithms for shortest paths in sparse networks,” Journal of the ACM, 24 (1): 1-13, 1977). All of these disclosures are incorporated by reference herein in their entirety. It is appreciated that other shortest path algorithms may also be possible. The shortest path algorithm may return the shortest distance between every two atoms in the molecular graph, with infinity to indicate unconnected atoms. In data representation, the infinite distances may be set to a value of −1, so that the network can see which pairs of atoms are not in the same molecule. This can also be extended to indicate atoms' membership to different molecules. For example, given a molecular graph containing 3 molecules: the system may set every pair of atoms between molecule #1 and #2 to a value of −1; set every pair of atoms between molecule #2 and #3 to −2; and set every pair of atoms between molecule #1 and #3 to −3.

The above described approach can be used to indicate membership in general. For example, given a molecular graph containing reactants, agents, and products, the system may set pairs of atoms between reactants and agents to a distance of −1, set pairs of atoms between agents and products to a distance of −2, etc. The above described atom features and bond features are only described as examples. It is appreciated that additional features may be added to improve the model performance, for example, in scenarios when the amount of training data is extremely limited.

In some embodiments, bond features may have two axis: an atom axis (which is atom feature) and a neighboring axis, where the diagonal is the atom seeing itself. Thus, bond features may contain information about every pair of atoms in a molecule, so the representation is a large square, which may be viewed as concatenating a row of atom representations for each atom in the molecule. The bonds in the molecule are indicated at locations in this “square” that correspond to specific pairs of atoms that are bonded. All other atom pairs in this matrix may be indicated by a “not bonded” feature. In the example described above, each element in the bond features representation may be a 45-dimensional vector.

In some embodiments, the described selection of input features may provide advantages over conventional neural network systems such as transformer neural network systems. For example, when a graph transformer network is used (e.g., in 106s in FIG. 1A), the positional encoding is provided as input features to the graph transformer work, rather than being added to a hidden layer of the transformer network. Additionally, and/or alternatively, the frequency coefficients of the positional embedding may be selected in such a way that floating-point overflow may be prevented. Any other suitable method for choosing a set of unique frequency coefficients may also be possible.

With further reference to FIG. 1A, the atom embedding layer 104 may be configured to perform a linear transformation over the atom features followed by an activation function to generate the atom representation (e.g., atom embedding). The linear transformation may refer to a matrix multiplication followed by a vector addition, such as: Ax+b where A is a matrix, b is a vector (e.g., a bias), and x is a vector. In some examples, the matrix A may have a dimension of 512×124, the atom feature may have a dimension of 124, and thus, the multiplication of Ax may result in a 512-dimensional vector. In some embodiments, the activation function may be an element-wise operation. In some examples, the activation function may a rectified linear unit (“ReLU”), for example, a max(x, 0) function. In this example, the activation function may set all negative values to zero. Other suitable activation function may also be used.

Additionally, the atom embedding layer 104 may be configured to normalize the atom features before performing the linear transformation such that no significant large values are provided to the linear transformation operation. For example, the normalization may include a symmetric log function: sign(x)*ln(abs(x)+1).

With further reference to FIG. 1A, bond embedding layer 102 may transform the bond features into bond representations. It's noted that all pairs of atoms have a representation. If the atom representations are thought of as a vector of representations, then the bond representations are a 2D matrix of representations. The dimensionality of the atom and bond representations need not be the same, and they may be a hyperparameter decided at the start of training.

In some embodiments, the bond embedding layer 102 may include a similar configuration as the atom embedding layer 104, except having a different dimension. For example, the bond embedding layer 102 may include a linear transformation (e.g., transforming bond features from 45-dimension to 64-dimension), followed by an activation function. Similar to atom embedding layer 104, the activation function in bond embedding layer 102 may also be a ReLU. Additionally, bond embedding layer 102 may be configured to normalize the bond features before performing the linear transformation such that no significant large values are provided to the linear transformation operation. For example, the normalization may include a symmetric log function: sign(x)*ln(abs(x)+1).

With further reference to FIG. 1A, the graph neural network 106 may include one or more layer(s) 106-1, . . . N each configured to update the atom representation (e.g., atom embedding). In some embodiments, the one or more layers 106 may be serially coupled and each layer is configured to update an input atom representation and input bond representation to generate an output atom representation and an output bond representation. In some embodiment, the input atom representation and input bond representation to the first layer in the graph neural network (e.g., 106-1) may be generated respectively by the atom embedding layer (e.g., 104) and bond embedding layer (e.g., 102). The output atom representation and output bond representation from the first layer (e.g., 106-1) may be provided as input to the second layer (e.g., 106-2), so on and so forth. As described above, the atom features contain information about the atoms, but not their neighbors; whereas bond features only contain information about each pair of atoms. Thus, in the layer(s) 106s in the graph neural network, information about the atoms and bond relationships are passed between atom features and bond features to update the atom representation and the bond representation. The updated atom representation from the last layer (e.g., 106-N) may be provided to the subsequent layers to be described below.

In some embodiments, the graph neural network (106s) may be a graph transformer network comprising one or more graph transformer layers that are serially coupled (as shown in FIG. 1A). FIG. 1D illustrates example components 120-1 of additional layers 120 in a neural network as shown in FIG. 1A, where the graph neural network is a graph transformer network. In some embodiments, the example components 120-1 may be implemented in the one or more additional layers 120 as shown in FIG. 1A.

With further reference to FIG. 1D, optionally, the components 120-1 may include an atom final layer 108 configured to pool the updated atom representations from the graph transformer layer (or the last graph transformer layer of serially coupled graph transformer layers) in FIG. 1A. In some examples, the atom final layer 108 may include a linear transformation. The atom final layer may project the atom representation obtained from the graph transformer network 106. In some examples, the input and output dimensions of the atom final layer may be the same, for example, 512. In some examples, the input and output dimensions of the atom final layer may be different. Additionally, the components 120-1 may include a summation operation layer 110 coupled to the atom final layer 108. In this configuration, the atom representation obtained from the graph transformer network may be pooled using a summation along the atom axis to generate a molecule representation, to be provided to the molecule layer (see 112 in FIG. 1A).

In some embodiments, the graph neural network (106s) may be a GCNN comprising one or more GCNN layers that are serially coupled (as shown in FIG. 1A). FIG. 1E illustrates example components 120-2 of additional layers 120 in a neural network as shown in FIG. 1A, where the graph neural network is a GCNN. In some embodiments, the example components 120-2 may be implemented in the one or more additional layers 120 as shown in FIG. 1A.

With further reference to FIG. 1E, the components 120-2 may include an atom final layer 128 configured to pool the updated atom representations from the graph neural network layer (or the last layer in the serially coupled GCNN) in FIG. 1A. In some embodiments, the atom final layer 128 may have a similar configuration as the atom final layer 108 (FIG. 1D). Additionally, the components 120-2 may include a bond final layer 126, which is configured in a similar manner as the atom final layer 128. Additionally, the components 120-2 may include a summation operation layer 130 coupled to the atom final layer 128 and bond final layer 126. In this configuration, summation operation layer 130 sums over the pooled atom representation from atom final layer 128 and the pooled bond representation from bond final layer 126 to generate a molecule representation according to which molecule each atom in the atom representation belongs. Comparing FIG. 1E to FIG. 1D, a difference is that the graph transformer network (FIG. 1D) uses only the final atom representation to compute final whole-molecule representation, whereas the GCNN (FIG. 1E) uses both the final atom and final bond representations.

Returning to FIG. 1A, molecule embedding layer 112 may be configured to perform one or more linear transformations over the output tensor from the summation operation 110, to generate a molecule representation. In some embodiments, the molecular layer 112 may include a feed-forward network, which may include one or more hidden layers (e.g., a first layer, a second layer etc), each followed by an activation layer (e.g., a first activation layer, a second activation layer etc.). In a non-limiting example, each hidden layer may include a linear transformation layer. For example, a first linear transformation layer may be configured to project the data from a first dimension (e.g., 512) to a second dimension (e.g., 1024). A second linear transformation may be configured to output the data in the same dimension as the input dimension (e.g., 1024). In some embodiments, the first and second activation layers each may be a ReLU. Other activation layers may also be possible.

In some embodiments, the target layer 114 may be configured to predict the properties of the input molecule based on the molecule representation (e.g., molecule embedding) from the molecule layer 112. For example, the target layer 114 may be configured to perform regression that outputs a mean and variance prediction for each target property. The target layer 114 may also be configured to perform classification that outputs a set of class probabilities. The details of the target layer 114 will be further described in the context of these operations with reference to FIGS. 1B and 1C.

FIG. 1B illustrates example components of a target layer 150 of a neural network for performing regression operation in predicting properties of molecules, according to some embodiments. For example, the target layer 150 may be implemented in the target layer 114 of the neural network 100 of FIG. 1A. In some embodiments, target layer 150 may include a first layer 152 configured to predict a value for at least one property based on the molecule representation (e.g., molecule embedding); and a second layer 154 configured to predict a deviation value for the at least one property based on the molecule representation. In some examples, the input to the first and second layers 152, 154 may be obtained from the neural network 100 of FIG. 1A, such as molecular layer 112. In some embodiments, the first layer 152 of the target layer may be configured to perform a linear transformation to project the molecule representation into a single output that represents a property value. In non-limiting examples, the property value may be any of a melting point, glass transition temperature, oxidation reduction potentials, flash point, etc.

It is appreciated that molecule property values may include boiling point, fluorescence quantum yield, UV/Vis/NIR absorption and emission spectra, CD spectra, NMR spectra, MS spectra, singlet triplet gap, chroma, color, hue, IR spectra, Raman spectra, Vibrational spectra, Quantum yields, Solubility, logP, ADME properties, blood-brain barrier penetration logBB, Reaction yield prediction, Reaction component classification, Synthetic accessibility score, Stoichiometry estimation, Molecule similarity estimation, Band gap, Orbital energies, Spin-orbit coupling, Charge, Energy, Reactivity, Toxicity, Stability, Lightfastness, Vapor pressure, Flammability, Flash point, Specific heat capacity, Thermal Conductivity, Electrical Conductivity, Viscosity, Density, or a combination of any of these properties. Other molecule properties or combination thereof may also be possible.

With further reference to FIG. 1B, the output of the target layer 150 may include a vector of numbers with dimensionality that depends on the dataset on which the neural network is trained. In the diagram, the regression target is a scalar value for the melting point. The target linear layer 152 converts the 1024 dimensional hidden representation into a 1 dimensional vector, which is interpreted as the predicted property value. In some embodiments, the single output may be a single value. In some embodiments, the single output may be multiple values, e.g., a vector. In the example shown in FIG. 1B, the output from the first layer 152 is a melting point of the input molecule.

With further reference to FIG. 1B, the second layer 154 of the target layer 150 may generate uncertainty value(s) associated with the value obtained from the first layer 152. For example, the uncertainty value(s) may be a standard deviation of the melting point obtained from the first layer 152. In some embodiments, the second layer 154 may be configured to perform a linear transformation to project the molecule representation into a single output that represents an uncertainty value, followed by an exponential operation, where yᵢ=e^{xi}. The purpose of the exponential operation in the uncertainty layer 154 is to constrain the predicted uncertainty values to be positive. In some embodiments, the second layer 154 may convert the hidden representation (e.g., in 1024-dimension) into the corresponding 1-dimensional variance. As shown in FIG. 1B, the output value of the second layer 154 may be a standard variance. In some embodiments, the system may generate the standard deviation by taking a square root of the predicted variance obtained from the second layer 154.

FIG. 1C illustrates example components of a target layer of a neural network for performing classification operation in predicting properties of molecules, according to some embodiments. In some embodiments, target layer 160 may be implemented in the target layer 114 of the neural network 100 of FIG. 1A. In some embodiments, target layer may include at least one layer, e.g., a target linear layer 162 configured to generate multiple properties simultaneously. For example, layer 162 may generate a plurality of values each indicating a likelihood of the input molecule belonging to a corresponding class of a plurality of classes.

In some embodiments, the plurality of classes may depend on the data provided. For example, the output could be the perceived color of the molecule: [red, orange, yellow, green, blue, purple]. In a non-limiting example, an output vector of [0.5, 0.1, 0.05, 0.5, 0.0, 0.0] may indicate a 50% probability that the molecule would appear red, a 10% probability that the molecule would appear orange, a 5% probability that the molecule would appear yellow, 5% probability that the molecule would appear green, 0% probability that the molecule would appear blue, and a 0% probability that the molecule would appear purple.

With further reference to FIG. 1C, the target linear layer 162 may be configured to perform a linear transformation followed by a softmax or Sigmoid activation. The linear transformation may project the molecule representation to a single output in a similar manner as the linear transformation operation in the target linear layer 152 of FIG. 1B. The use of softmax or Sigmoid activation may depend on the data used and the properties that need to be predicted. For example, the target linear layer 162 may use the softmax activation when the classes are mutually exclusive (e.g., a molecule cannot appear to be red and green, it must be one or the other). An example of a softmax activation may be expressed as:

$$softmax ( {x}_{i} ) = {exp}^{( xi )} / {\sum }_{j} {exp}^{( xj )}$$

Thus, a softmax activation may be used to enforce that the probabilities are summed to 1. This is referred to as a multinomial logistic regression. In some embodiments, the target linear layer 162 may use a sigmoid activation when the output classes are independent (e.g., melting point above/below 300K and planar/nonplanar). This is referred to as a binary logistic regression.

As described above, the target layer of the neural network (e.g., 114 of FIG. 1A) may be configured to perform various tasks, such as regression or classification tasks, where a regression task generates predicted property value(s) of the input molecule and a classification task generates a one or more probability values of the input molecule belonging to a class of a plurality of classes. Thus, the configuration of the graph neural network (e.g., 106 of FIG. 1A, which may be a graph transformer network or a GCNN) may also vary depending on the prediction tasks performed. The details of the graph neural network are further described with reference to FIGS. 2-7B.

FIG. 2 illustrates an example graph transformer layer 200 of a neural network for performing regression operation in predicting properties of molecules, according to some embodiments. In some embodiments, graph transformer layer 200 may be implemented in a graph transformer neural network. For example, graph neural network 106s may be a graph transformer neural network and graph transformer layer 200 may be implemented in any of the neural network layers 106-1, . . . N of neural network 100 (FIG. 1A). As shown in FIG. 2, the input to the graph transformer layer 200 may include atom representation (e.g., atom embedding) and bond representation (e.g., bond embedding). The atom representation and the bond representation may be provided from the atom embedding layer (e.g., 104 of FIG. 1A) and the bond embedding layer (e.g., 102 of FIG. 1A), respectively. Alternatively, the atom representation and the bond representation may be provided from output of another graph transformer layer (e.g., one of the layers 106-1, . . . N).

As shown in FIG. 2, in some embodiments, graph transformer layer 200 may include a first residual connection 250 configured to update the bond representation with the atom representation. The first residual connection 250 may include a path from the input atom representation to intersect the input bond representation at operation 210. In some embodiments, operation 210 may be configured to perform a summation operation, such as an element-wise summation, to update the bond representation based on the atom representation. The element-wise summation operation in operation 210 may result in a saving of computation because the atom representation and bond representation are compressed to form a combined data in a reduced amount (e.g., relative to concatenation of the atom representation and bond representation).

With reference to FIG. 2, the graph transformer layer 200 may include one or more blocks (e.g., 202, 204, 206, 208) to process the atom representation. In some embodiments, the graph transformer layer 200 may include an atom linear layer 202 and neighbor linear layer 204 each respectively configured to perform a linear transformation over the atom representation. The atom representation (e.g., atom embedding) may include data in atom axis and neighbor axis, where the diagonal elements represent the atoms themselves. In some examples, each of the atom linear layer 202 and neighbor linear layer 204 may be configured to project the atom representation from 512-D to 64-D. In some embodiment, the graph transformer layer 200 may further include operations 206, 208 each respectively configured to form updated atom representations.

In some embodiments, operation 206 may be configured to repeat the output data from the atom linear layer 202 along the neighbor axis. For example, operation 206 may duplicate the output of atom linear layer 202 once for each atom, stack duplicates in a new neighbor axis (inserted between the existing two axes) so that the tensor is of size [atom_count, atom_count, bond_hidden_dim], where bond_hidden_dim is the feature dimensionality (e.g., 64 or other suitable values). In some embodiments, operation 208 may be configured to repeat the output data from the neighbor linear layer 204 along the atom axis. For example, operation 208 may duplicate the output of neighbor linear layer 204 once for each atom, stack duplicates in a new atom axis (inserted before the existing two axes) so that the tensor is of size [atom_count, atom_count, bond_hidden_dim], where bond_hidden_dim is the feature dimensionality (e.g., 64 or other suitable values). The updated atom representations from the operations 206, 208 may be provided to operation 210 for updating the bond representation (e.g., bond embedding).

With further reference to FIG. 2, graph transformer layer 200 may further include an attention network 252 coupled to the first residual connection. The attention network 252 may be configured to ignore (zero out) the unrelated (unbounded) atoms that should not be propagated. In some embodiments, the attention network 252 may include one or more blocks 212-224, and may be configured to use the input atom representation and the updated bond representation from the first residual connection (e.g., output of operation 210) to generate output. The output may be the input atom representation updated based on the updated bond representation. Considering a large molecular may include many bonding relationships (e.g., reflected in bonding representation), although there might be only a few atoms that are bonding. The attention network is configured to determine what bonding relationship is relevant (indicating bonding atoms) and zero out the bonding relationships that are not relevant.

In some embodiments, the attention network 252 may include an attention layer 212 configured to predict one or more attention scores based on the updated bond representation. Attention scores are weighted averages and they reflect what message (between neighboring atoms) is relevant and what is not relevant. Low scores may be used to zero out other atoms that are not bonded. In some embodiments, the attention network 252 may include a message layer 214 configured to generate one or more bond messages based on the updated bond representation (e.g., output from block 210), where the bond messages reflect the messages of neighboring atoms. A bond message may be a bond representation that describes information about the pairs of atoms. When the attention scores are applied to bond messages, messages that represent unbonded relationships between pairs of atoms (e.g., having low scores) should not be propagated, whereas messages that represent bonded relationships (e.g., having high scores) should propagate. This attention mechanism in the graph transformer network enables the system to focus on important information and enables the network to converge faster.

With further reference to FIG. 2, the attention layer 212 and message layer 214 are further described in detail. In some embodiments, the attention layer 212 may be configured to perform a linear transformation followed by a softmax activation. In some examples, the linear transformation may project the bond representation from block 210 (e.g., bond embedding) from one dimension (e.g., 64-D) to another dimension (e.g., 4-D). The softmax may be performed along the neighbor atom axis to generate the attention scores. In some embodiments, the graph transformer network may be implemented in a message passing neural network in that the message layer 214 may calculate messages among the atoms. In a non-limiting example, the message layer 214 may be configured to perform a linear transformation to generate bond messages. The linear transformation in message layer 214 may be configured to project the bond representation (e.g., output from block 210) in a dimension (e.g., 64-dimension) to another dimension (e.g., 16-dimension) to form the bond messages. In some embodiments, the attention network may further include operations, 216, 218. For example, operation 216 may duplicate attention scores tensor message_dim times, where message_dim is the dimensionality of the bond messages. Operation 216 may further stack the duplicates in a new attention axis (inserted between the second and third axes) so that the tensor is of size [atom_count, atom_count, attention_heads, message_dim], where attention_heads is the number of individual attention heads (e.g., 4-D or other suitable dimensions), and message_dim is the dimensionality of the messages (e.g., 16-D or other suitable dimensions).

Similarly, operation 218 may duplicate bond messages tensor attention_heads times, where attention_heads is the number of individual attention heads. Operation 218 may further stack the duplicates in a new message axis (inserted after the third axis) so that the tensor is of size [atom_count, atom_count, attention_heads, message_dim], where attention_heads is the number of individual attention heads (e.g., 4-D or other suitable dimensions), and message_dim is the dimensionality of the messages (e.g., 16-D or other dimensions).

In some embodiments, the graph transformer layer 200 may be configured to control the extent of bond message passing through the neural network via attention scores. For example, if the graph transformer network is learned to always return an attention score of zero for nonbonded atoms and a output of one for all bonded atoms (e.g., before softmax activation), then the result would be a message passing network which effectively performs an averaging operation when aggregating the messages.

With further reference to FIG. 2, the attention network 252 may be configured to combine the one or more attention scores (e.g., obtained from attention layer 212) and the one or more bond messages (e.g., obtained from message layer 214) by a multiplication operation 220, such as an element-wise multiplication. In some examples, the attention scores may be generated such that they are summed to 1. When the attention scores are applied to the bond messages via the element-wise multiplication operation at block 220, this operation effectively propagates the messages for atoms that are not bonded and calculates a weighted average of the bond representation.

With further reference to FIG. 2, the attention network 252 may include a summation operation 222. For example, summation operation 222 may sum along neighbor axis by taking a tensor of shape [atom_count, atom_count, attention_heads, message_dim](e.g., output from the multiplication operation 220) and returning a tensor of shape [atom_count, attention_heads, message_dim]. The summation operation 222 may reduce the dimension of the output tensor from operation 220 to an updated representation. In some embodiments, the attention network may also include a merge operation 224 configured to merge attention and feature axes. In a non-limiting example, merge operation 224 may reshape tensor to combine attention and message axes into a new feature axis. This operation may take a tensor of shape [atom_count, attention_heads, message_dim] and return a tensor of shape [atom_count, atom_embedding_update_dim] where atom_embedding_update_dim is the product of attention_heads and message_dim (e.g., 4*16=64 in the above example).

With further reference to FIG. 2, in some embodiments, the attention network 252 may include one or more additional operational blocks to generate the output. For example, the attention network may 252 further include concatenation operation 226 to perform an operation that combines the input atom representation and the updated bone representation from the attention network described above. In some examples, operation 226 may be a concatenation of data along feature axis. Thus, the output of the attention network may be the updated bond representation further updated based on the input atom representation. In a non-limiting example, concatenation operation 226 may be operated to concatenate atom embedding (e.g., from input of the graph transformer layer 200) and atom embedding update tensors (e.g., from the attention network) in the feature axis. This operation may result in a tensor of shape [atom_Count, atom_hidden_dim+atom_embedding_update_dim]. In this example, the dimensions of all axes other than the feature axis match exactly for all input tensors.

With further reference to FIG. 2, additionally and/or alternatively, the graph transformer network 200 may include an atom feed-forward network (FFN) 228 to generate a new atom representation based on the output of the attention network 252, where the new atom representation may be provided as output of the graph transformer layer 200. In a non-limiting implementation, the atom FFN 228 may include serially coupled neural network layers. For example, atom FFN 228 may include multiple linear layers configured to perform a series of linear transformations, each followed by an activation function. For example, the atom FFN 228 may include a first linear transformation followed by a first activation function; and a second linear transformation followed by a second activation function. The first linear transformation may be configured to project the output tensor from the attention network in a first dimension (e.g., 1) to a second dimension (e.g., 1024). The second linear transformation may be configured to project the output tensor from the first linear transformation (e.g., in 1024-dimension) to another dimension (e.g., 512). In some examples, the first and second activation functions may each be a ReLU. Other activation layer may also be possible.

In some embodiments, the graph transformer layer 200 shown in FIG. 2 may be repeated in the graph transformer network (e.g., 106 of FIG. 1A). Having multiple graph transformer layers serially connected may create a deeper neural network that iteratively updates the atom and bond embeddings. In a non-limiting example, four graph transformer layers may be serially connected. These stacking layers increase representational power quicker than simply increasing the number of neurons in a single layer. Stacking layers also allows the network to learn hierarchical representations. Any other suitable number of graph transformer layers, for example, two layers, six layers, or other suitable number of layers may be used. More layers may indicate increased capacity of the model and more computational resources that are needed to run the neural network. As such, an optimal number of graph transformer layers may be used.

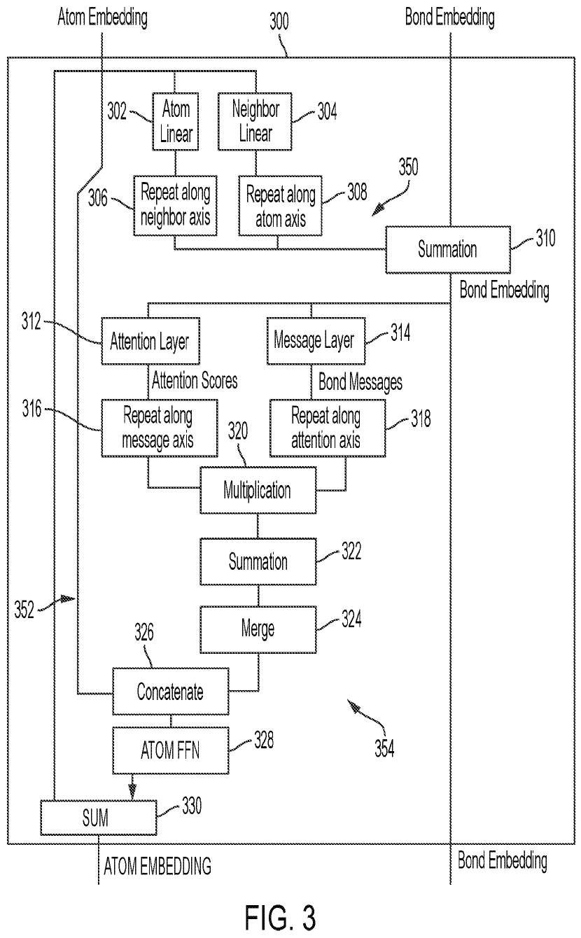

FIG. 3 illustrates an example graph transformer layer of a neural network for performing classification operation in predicting properties of molecules, according to some embodiments. In some embodiments, graph transformer layer 300 may be implemented in any of the graph transformer layers 106-1, . . . N of neural network 100 (FIG. 1A). As shown in FIG. 3, the input to the graph transformer layer may include atom representation (e.g., atom embedding) and bond representation (e.g., bond embedding). Alternatively, the atom representation and the bond representation may be provided from output of another graph transformer layer (e.g., one of the layers 106-1, . . . N).

The graph transformer layer 300 may be configured to operate in a similar manner as graph transformer layer 200 of FIG. 2, except that graph transformer layer 300 may include an additional residual connection. Accordingly, the components having reference numerals 302-328 in FIG. 3 may be similar components having reference numerals 202-228 in FIG. 2. The graph transformer layer 300 may also include a residual network 350 similar to residual network 250 in FIG. 2. The graph transformer layer 300 may also include an attention network 352 similar to attention network 252 in FIG. 2. Thus, the detailed descriptions of these various components in graph transformer layer 300 are not repeated.

Additionally, the graph transformer layer 300 may include the additional residual connection 354 coupled to the attention network 352 and configured to update the input atom representation with the updated atom representation from the attention network 352. As shown in FIG. 3, the additional residual connection 354 may include a path from the input atom representation through the first residual connection 350 and the attention network 352 (similar to the attention network described in FIG. 2) to atom FFN 328 and intersect with the input atom representation at operation block 330. In some embodiments, operation 330 may include an element-wise sum operation between output of atom FFN layer 328 and atom embedding (as input of the graph transformer layer 300). The element-wise operation may require the shape of the input tensors to exactly match. For example, the two input tensors to the element-wise summation (as well as the output tensors) have the shape [atom_Count, atom_hidden_dim]. Thus, the additional residual connection 354 effectively updates the input atom representation with the updated atom representation (from the attention work 352).

Additionally, and/or alternatively, in graph transformer layer 300, the atom FFN layer 328 may be configured similar to the atom FFN layer 228 of FIG. 2 except atom FFN 328 may not need the last activation layer. For example, the atom FFN layer 328 may include a first linear transformation followed by a first activation layer, which is followed by a second linear transformation layer. Such configuration for the additional residual connection may improve the performance of the transformer.

As described above, all neural network layers in various network configurations perform a feature to feature transformation, meaning that they do not directly propagate information between atoms or bonds. Rather, each network layer only changes the feature dimension of a representation. For example, atom embedding layer (e.g., 202, 302 in FIGS. 2 and 3) change the atom features tensor of shape [atom_count, atom_feature_dim] to the atom embedding [atom_count, atom_hidden_dim], where atom_hidden_dim is the dimensionality of the hidden representation for the atoms (e.g., 512-D or other suitable dimensions). In the network configurations described herein, the shape or rank of a tensor may be changed via operations in the network, such as operations 206/306, 208/308, 216/316, 218/318, 220/320, 222/322, 224/324, 226/326, and 330 (in FIGS. 2 and 3).

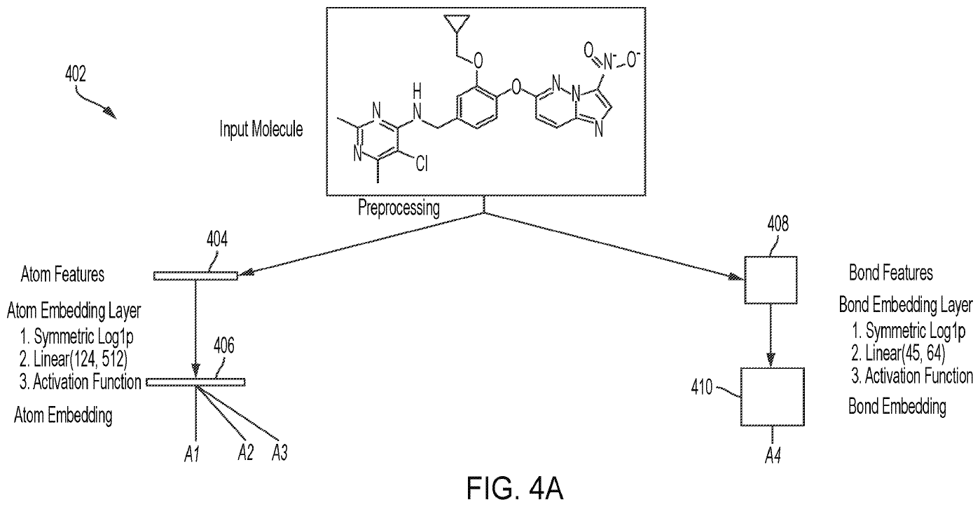

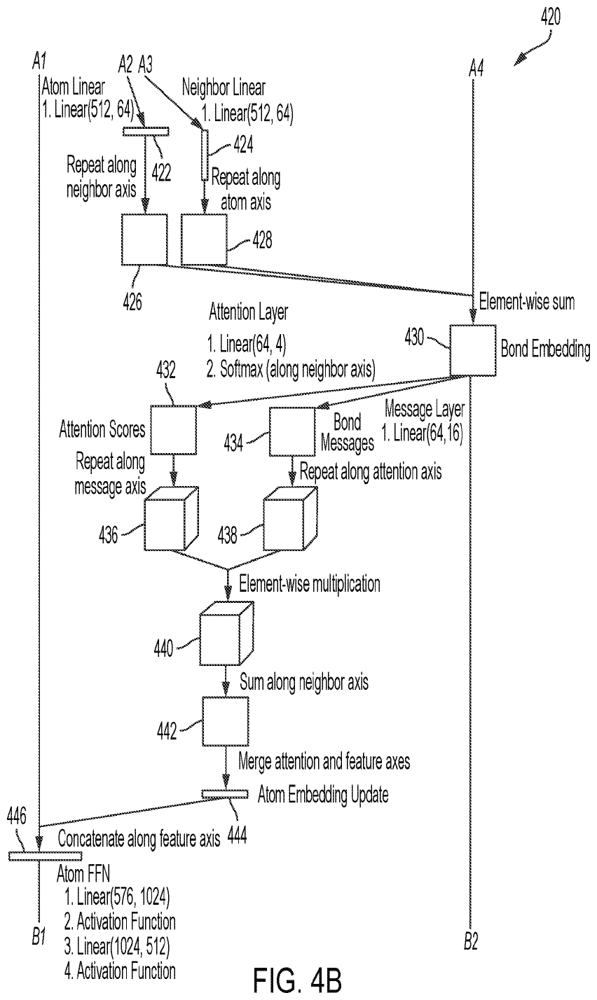

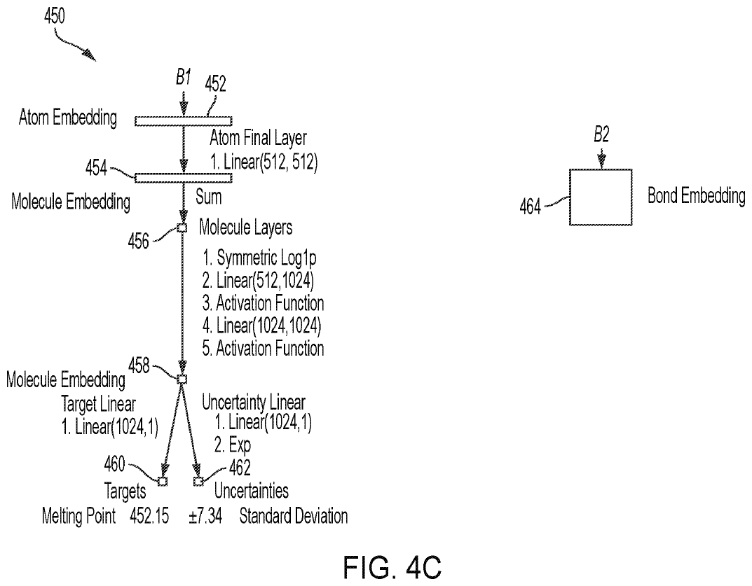

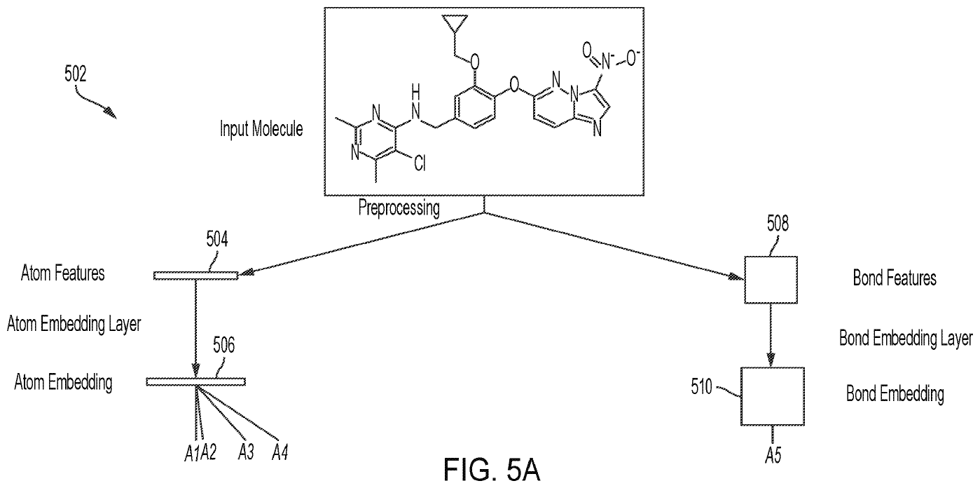

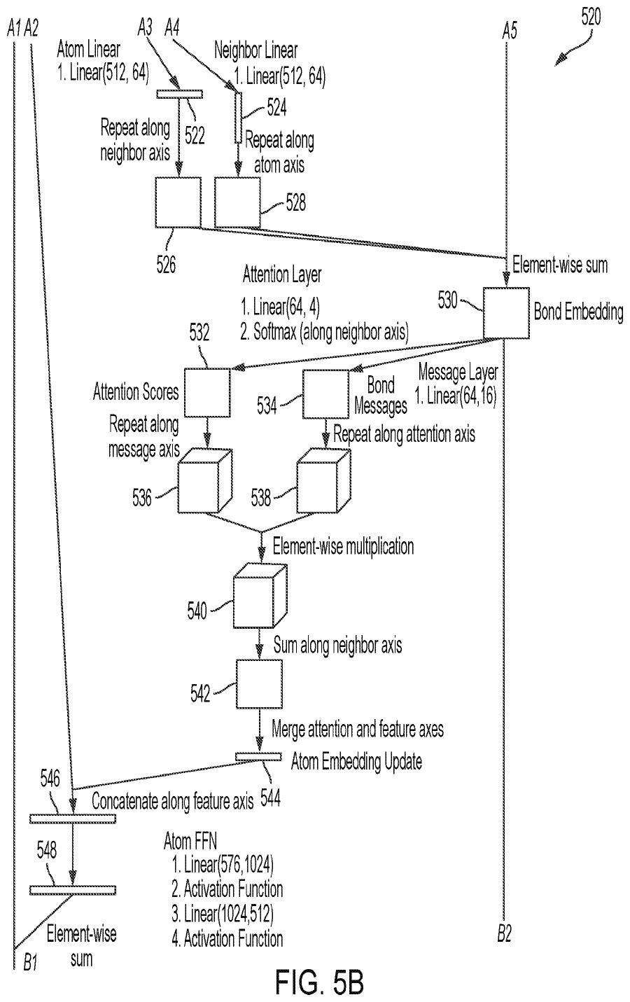

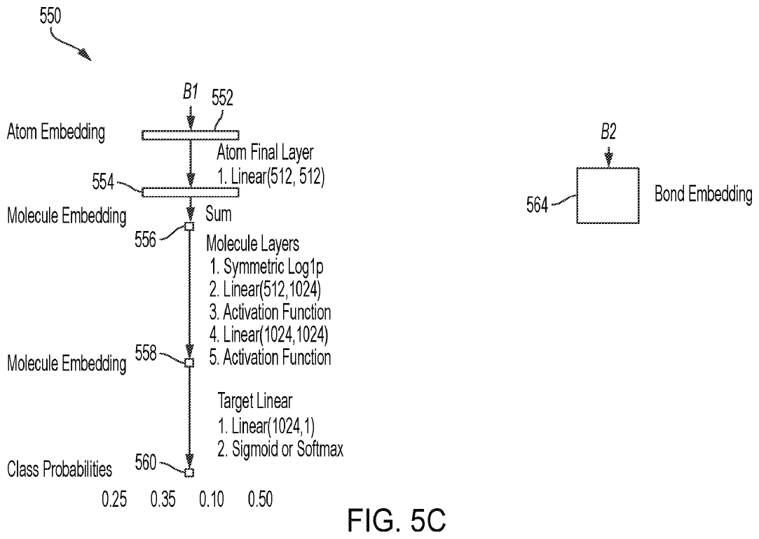

FIGS. 4A-5C illustrate various components of the neural network 100 (FIG. 1A) with the notion of input and output dimensionalities. For example, FIGS. 4A-4C illustrate portions of an implementation of a neural network 100 (FIG. 1A) for performing regression operation in predicting properties of molecules, according to some embodiments. For example, the portion of the neural network 402 shown in FIG. 4A may implement layers 102, 104 of FIG. 1A, where boxes 404, 406 correspond to the input and output of atom embedding layer (104 in FIG. 1A), respectively; and boxes 408, 410 correspond to the input and output of bond embedding layer 102 (FIG. 1A), respectively.

With reference to FIG. 4B, the portion of the neural network 420 may implement various components of graph transformer network layer(s) 200 in FIG. 2. For example, boxes 422, 424 correspond to the output of atom linear 202 and neighbor linear 204 (FIG. 2), respectively. Boxes 426, 428 correspond to the output of boxes 206, 208 (FIG. 2), respectively. Box 430 corresponds to the output of summation operation 210 (FIG. 2). Boxes 432, 434 correspond to the output of boxes 212, 214 (FIG. 2), respectively. Boxes 436, 438 correspond to the output of boxes 216, 218 (FIG. 2), respectively. Box 440 corresponds to the output of multiplication 220 (FIG. 2). Box 442 corresponds to the output of summation operation 222 (FIG. 2). Box 444 corresponds to the output of merge operation 224 (FIG. 2). Box 446 corresponds to the output of concatenation operation 226 (FIG. 2).

With reference to FIG. 4C, the portion of the neural network 450 may implement layers/operations 108, 110, 112, 114 in FIG. 1A, where boxes 452, 454 correspond to the input and output of atom final layer 108 (FIG. 1A), respectively; box 456 corresponds to the output of summation layer 110 (FIG. 1A); box 458 corresponds to the output of molecular layer 112 (FIG. 1A); and boxes 460, 462 correspond to the output of target layer 114 (FIG. 1A) and also correspond to the output of target linear 152 and uncertainty linear 154 (FIG. 1B), respectively.

FIGS. 5A-5C illustrate portions of an implementation of a neural network 100 (FIG. 1A) for performing classification operation in predicting properties of molecules, according to some embodiments. For example, the portion of the neural network 502 shown in FIG. 5A may implement layers 102, 104 of FIG. 1A, where boxes 504, 506 correspond to the input and output of atom embedding layer (104 in FIG. 1A), respectively; and boxes 508, 510 correspond to the input and output of bond embedding layer 102 (FIG. 1A), respectively.

With reference to FIG. 5B, the portion of the neural network 520 may implement various components of graph transformer network layer(s) 300 in FIG. 3. For example, boxes 522, 524 correspond to the output of atom linear 302 and neighbor linear 304 (FIG. 3), respectively. Boxes 526, 528 correspond to the output of boxes 306, 308 (FIG. 3), respectively. Box 530 corresponds to the output of summation operation 310 (FIG. 3). Boxes 532, 534 correspond to the output of boxes 312, 314 (FIG. 3), respectively. Boxes 536, 538 correspond to the output of boxes 316, 318 (FIG. 3), respectively. Box 540 corresponds to the output of multiplication 320 (FIG. 3). Box 542 corresponds to the output of summation operation 322 (FIG. 3). Box 544 corresponds to the output of merge operation 324 (FIG. 3). Box 546 corresponds to the output of concatenation operation 326 (FIG. 3).

With reference to FIG. 5C, the portion of the neural network 550 may implement layers/operations 108, 110, 112, 114 in FIG. 1A, where boxes 552, 554 correspond to the input and output of atom final layer 108 (FIG. 1A), respectively; box 556 corresponds to the output of summation layer 110 (FIG. 1A); box 558 corresponds to the output of molecular layer 112 (FIG. 1A); and boxes 560 corresponds to the output of target layer 114 (FIG. 1A) and also corresponds to the output of target linear 162 (FIG. 1C).

In FIGS. 4A-5C, the various linear operations are represented with the notion of input and output dimensionalities. For example, with reference to FIG. 4B, operation Linear(576, 1024) accepts a 576 dimensional input vector and generates a matrix, which is multiplied with Linear (1024, 512)—a weight matrix of shape 1024×576, resulting in a 1024 dimensional vector 452 as output of the atom final layer (108 in FIG. 1A). In these figures, various data representations are represented by rectangle boxes, which correspond to tensors of the neural network. In the neural network configurations described herein, the arrows correspond to operations (by one or more components in the network as described in FIGS. 1A, 1B, 2, and 3), which form the links between tensors. A small square box in the diagram may represent a single object, such as a molecule at output of the summation operation (e.g., boxes 456 of FIG. 4C). The slender rectangular box may represent data having multiple elements. For example, atom representations are represented by a slender rectangle of the same height to indicate that there are multiple atoms in a given molecule (e.g., boxes 436, 438, 440 in FIG. 4B; boxes 536, 538, 540 in FIG. 5B). One can view this as concatenating multiple atoms (represented as small black squares) together into a single tensor (this forms a rectangle).

With further reference to FIGS. 4A-5C, dimensionalities of various data/tensor flow may be represented by the sizes of the boxes, although the sale may not be accurate and may be illustrative only. For example, the initial shape of Atom Features 404 (FIG. 4A), 504 (FIG. 5A) may be [atom_count, atom_feature_dim], where atom_count is the number of atoms in a given molecule, and atom_feature_dim is the feature dimensionality of each atom (e.g., 124 in the example previously described). The initial shape of Bond Features 408 (FIG. 4A), 508 (FIG. 5A) may be [atom_count, atom_count, bond_feature_dim], where atom_count is the number atom atoms in a given molecule, and bond_feature_dim is the feature dimensionality of each bond (e.g., 45 in the example previously described). The bond feature tensor 408, 508 may contain two axes of length atom_count which represent every pair of atoms. The first axis may be the atom axis, which has the same meaning as the first axis of the atom feature tensor. The second axis of the bond feature tensor may be the “neighbor axis”. For example, to index along this axis, a specific neighbor is selected. This is analogous to indexing along the atom dimension, by selecting a specific atom.

The meanings assigned to a particular axis may be useful for understanding high rank tensors and how information moves through the network. In some embodiments, the atom and bond representations of multiple molecules may be batched, meaning that there's also a leading “batch” axis as well, where a batch size of 1 may be a single molecule.

With further reference to FIGS. 4A-5C, the bond features must contain information about every pair of atoms in a molecule, so the bond features may be represented in a large square. One can view this as concatenating a row of atom representations for each atom in the molecule (this what generates a square). The bonds in the molecule are indicated at locations in this “square” that correspond to specific pairs of atoms that are bonded. All other atom pairs in this matrix are indicated by a “not bonded” feature.

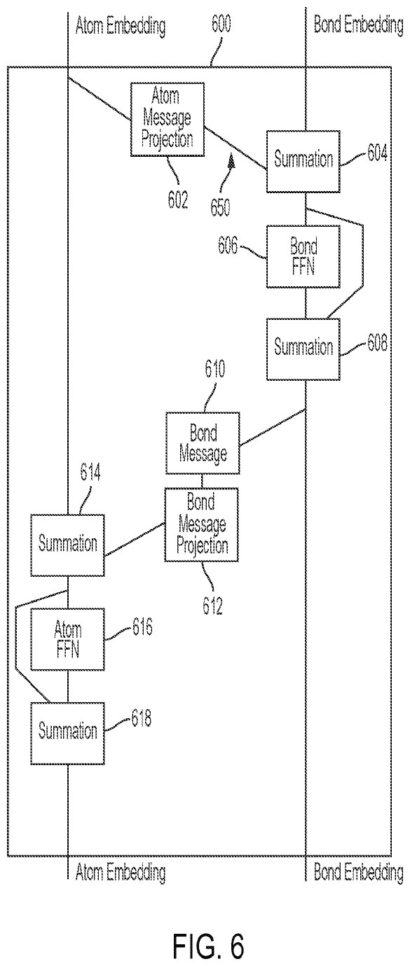

FIG. 6 illustrates an example GCNN layer 600 of a neural network for predicting properties of molecules, according to some embodiments. In some embodiments, the graph neural network (e.g., 106s in FIG. 1A) may be a GCNN. In other words, GCNN layer 600 may be implemented in any of the neural network layers 106-1, . . . N of neural network 100 (FIG. 1A). As shown in FIG. 6, the input to the GCNN layer 600 may include atom representation (e.g., atom embedding) and bond representation (e.g., bond embedding). The atom representation and the bond representation may be provided from the atom embedding layer (e.g., 104 of FIG. 1A) and the bond embedding layer (e.g., 102 of FIG. 1A), respectively. Alternatively, the atom representation and the bond representation may be provided from output of another GCNN layer (e.g., one of the layers 106-1, . . . N). Thus, when the GCNN layers (e.g., multiple instances of layer 600) are serially coupled (as shown in 106s in FIG. 1A), the GCNN takes initial atom and bond embeddings and updates each of them before outputting a final atom and bond embedding.

With further reference to FIG. 6, in GCNN layer 600, all of the atoms embedding representations are stacked together in a batch (e.g., number of atoms in the batch×atom features) and an index is maintained. The atom representations are transformed with one or more neural network layers (e.g., 2 or other suitable number) and non-linearities, then added to the bond representations of the bonds each atom is connected to. Bond features are then transformed by one or more fully connected neural network layers. These bond features are then used to update the atom features of the atoms the bonds are connected to. The atom and bond representations are then sum-pooled according to which molecule each atom in the batch belongs to generate molecule representations for further processing. The details of a GCNN layer 600 is further explained.

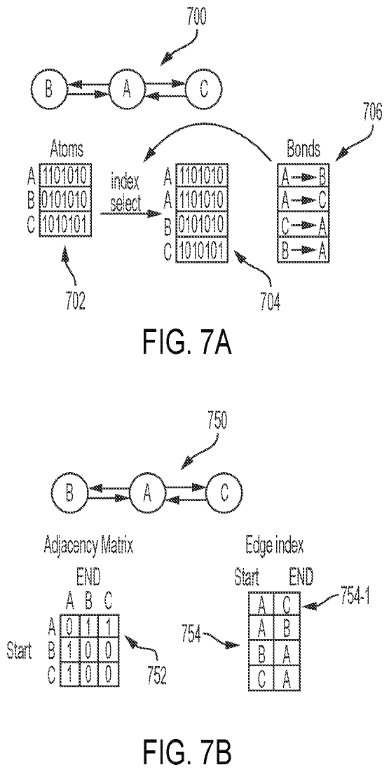

In some embodiments, GCNN layer 600 may include a similar structure as graph transformer layer 200 but does not include the attention network. For example, GCNN layer 600 may include atom message projection 602 configured to project the atom embedding in a similar manner as network layer in the graph transformer network (e.g., 200 in FIG. 2). In some embodiments, atom message projection 622 may include a multiple-layer neural network (e.g., three layer feed forward neural network, or other suitable neural network) followed by an index-select operation. The feed forward neural network portion non-linearly transforms the atom representations to form updated atom representations. The index-select operation indexes the new atom representation list according to which bond(s) of which each of the atoms is a part. FIG. 7A illustrates an example of index-select operation, according to some embodiments. As shown in FIG. 7A, a graph 700 is defined by 3 nodes and 4 directional edges. Index-select operation may take the edge list and then repeat certain node representations based on how many edges they have. As shown, node A is repeated in the updated atom representation 704 because node A has more edges than nodes B and C. The updated atom representation 704 can then be added to bond representation, e.g., via a summation operation 604 (FIG. 6).

Returning to FIG. 6, GCNN layer 600 may further include a bond FFN 606, which may have a similar structure as atom FFN (e.g., 228 in FIG. 2). In a non-limiting example, the bond FFN 606 may be a two-layer FFN and configured to project the output from the summation operation 604 to another dimension. Network layer 600 may further include another summation operation 608 configured to sum the output of the bond FFN 606 and the output of the summation operation 604. Similar to other summation operations in network layer 200, 300 (FIGS. 2 and 3), summation operation 604 and 608 may perform element-wise summation in that the atom representation and bond representation are compressed to form a combined data in a reduced amount.

As shown in FIG. 6, the configuration of summation operations 604, 608 with the bond FFN 606 coupled in between forms a residual connection, in which a bond representation (e.g., output from summation operation 604) is transformed using a neural network (e.g., bond FFN 606) and then the transformed representation (e.g., output of bond FFN 606) is added to the original representation (e.g., output of the summation operation 604) to generate the output (e.g., from summation operation 608). This output is an updated bond representation that has some characteristics from the transformed representation and some characteristics from the un-transformed representation.

With further reference to FIG. 6, GCNN layer 600 may further include a bond message operation 610 configured to generate one or more bond messages (e.g., as described in 214 in FIG. 2) based on the updated bond representation (e.g., output from block 608). In a non-limiting example, the bond message operation 610 may be a one-layer neural network, or any other suitable network.

In some embodiments, GCNN layer 600 may further include a bond message projection 612 configured to project the bond messages from box 610 to another dimension. In some examples, the bond message projection 612 may include a scatter-add operation followed by a FFN (e.g., three-layer FFN). The scatter-add operation is the inverse of the index-select described above (e.g., FIG. 7A), which generates bond representations, whereas the FFN pools the bond representations according to which atoms each bond is connected to.

With further reference to FIG. 6, GCNN layer 600 may further include summation operation 614 coupled to atom FFN 616 and summation operation 618. Atom FNN 616 may have a similar structure as atom FNN 228, for example. In a non-limiting example, atom FNN 616 may be a two-layer neural network. Summation operations 614, 618 may be configured in a similar manner as summation operations 604, 608 described above. For example, summation operations 614, 618 may form a residual connection under which an atom representation (e.g., output from summation operation 614) is transformed using a neural network (e.g., atom FFN 616) and then the transformed representation (e.g., output of atom FFN 616) is added to the original representation (e.g., output of the summation operation 614) to generate the output. This output is an updated atom representation that has some characteristics from the transformed representation and some characteristics from the un-transformed representation.

As shown in FIG. 6, the GCNN (e.g., layer 600) does not use attention network as in graph transformer network (e.g., layer 200). Further, a sparse GCNN may be implemented, where the connectivities of the atoms are stored using a sparse matrix. FIG. 7B illustrates an example of sparse matrix for representing atom connectivities, according to some embodiments. In classical graph information theory, a graph 750 defines the atom connectivities in nodes and edges, similar to graph 700 (FIG. 7A). Bonds may be represented using an adjacency matrix 752, which is a square matrix with a row and a column for each node. In spare matrix representation 754, atom connectivities are represented by edge indices, which include a list of pairs, one for each bond. Each pair (e.g., 754-1) includes a start atom (e.g., A) and an end atom (e.g., C) for that bond. Comparing the two representations 752, 754, the adjacency matrix needs (number of atoms)\hat{ }2 elements, whereas the sparse matrix needs 2× (number of bonds) elements. Consider the spart matrix stores only the bonds that do in fact exist, sparse matrix representation may result in significant saving in memory space and improved efficiency of subsequent processing. In some examples, chemical graphs may tend to have relatively smaller graphs and lower number of bonds per atom, which may result in a greater gain from sparse matrix representation.

As shown above, in sparse GCNN, the network runs faster because only existing bonds are accounted for, so that the model has higher molecule/second throughput. Further advantages over conventional system include space efficiency as the molecules are packed into batches that do not require any padding because batches of compounds is viewed as one giant disconnected graph. Further advantages include accuracy improvement. For example, in some scenarios, sparse GCNN outperforms the graph transformer. In some embodiments, the GCNN layer 600 in FIG. 6 may be adapted to perform both regression and classification. For example, GCNN layer 600 may be configured to use RELU activation for regression as opposed to using CELU activation for classification. The individual neural networks in GCNN layer 600 may also be configured differently. For example, for classification, ortho-linear layers may be used in the embedding, whereas linear layers may be used for regression.

As illustrated in various embodiments in FIGS. 1A-7B, the systems and methods for predicting molecule properties may selectively use the graph transformer network (see FIGS. 2-5C) or GCNN (see FIGS. 6-7B) depending on the performance of the two networks, which may vary depending on the nature of the problem.

In some embodiments, various training techniques may be used to train the parameters/weights of various layers in the neural network described herein (e.g., FIG. 1A-7B). In some embodiments, prior to the start of training, the mean and variance of regression targets are estimated and used to adjust the initialized weights and biases of the final layer of the network such that the network outputs target values with the estimated mean and variance of the targets, on average. The expected output of a neural network with proper initialization is a mean of zero and a variance of 1, on average. This is effectively the same as standardizing the targets by subtracting their mean and dividing by their variance, except this approach avoids the need to transform the outputs of the network back into the right mean and variance afterward. Such approach may improve convergence of the training since the network starts much closer to a minimum on the loss surface and does not need to dedicate neurons to this information over the course of training. It also reduces the effect of regression target magnitude on training dynamics, leading to better training stability on a wider variety of datasets.

In some embodiments, during training, the learning rate is warmed up slowly to minimize the chance of divergence. In some embodiments, the calculated gradients are clipped with a similar method to the one described in Seetharaman, Prem, et al. “Autoclip: Adaptive gradient clipping for source separation networks.” 2020 IEEE 30th International Workshop on Machine Learning for Signal Processing (MLSP). IEEE, 2020, APA, which is incorporated by reference herein. In a non-limiting example, the clipping percentile may be 10%. Other suitable values may also be possible.

In some embodiments, the training system may store most recent number of gradient norms to save computing resource because the growing history of gradient norms becomes increasingly expensive to store and compute the percentile thereof as training progresses. For example, the system may store about the most recent 10,000 gradient norms. Other suitable number may also be possible.

In some embodiments, the system may interpolate between a clipping value of 1 and the calculated clipping value as the gradient norm window is filled. This stabilizes the clipping value at the very start of training when the history length is short. During training, the loss function may ignore model predictions for missing regression targets when calculating the loss. This may allow the system to utilize more data, since not every target for each example needs to be known.

In some embodiments, the loss contribution of each regression target may be automatically balanced based on the number of missing values. For example, the training process may keep track of the number of missing target values encountered during the training and scale the contribution of each regression target accordingly. This is to avoid a situation where the model focuses on performing well on targets which have more data, at the expense of targets with more missing values.

In some embodiments, the magnitude of the loss contribution between regression targets may be balanced dynamically using running averages of the per target losses. Targets with relatively large variances tend to dominate the loss during training, which can cause the model to focus on them rather than targets with relatively low variance. Accordingly, the use of the running averages of the per target losses may mitigate this effect.

The various embodiments described above with reference to FIGS. 1A-7B may be advantageous over conventional systems. For example, the system described above uses a graph transformer network, which includes an attention network to propagate information between atom and bond representations. This facilitates the use of simple input features, limiting the need for time-consuming feature engineering when applying the model to new properties, and thus, make the network more efficient. The GCNN configuration (e.g., FIGS. 6-7B) may include similar advantages, and or other advantages as described herein with respect to FIGS. 6-7B.

Additionally, for both classification and regression networks, the graph transformer network uses a residual connection to update the bond representation based on the atom representation. This improves the efficiency of the network because the updated bond representation results in a saving of data in comparison to the conventional approach of concatenating the bond representation and atom representation.

Additionally, in the classification network, the graph transformer network includes an additional residual connection after the attention network, to add the new atom representation back to the input atom representation, which improves the performance of the transformer.

Additionally, the use of shortest path features in the input bond features may improve the graph transformer network's understanding of the structure of the molecular graph in the first few layers, allowing the system to use shallower (and thus faster) neural networks.

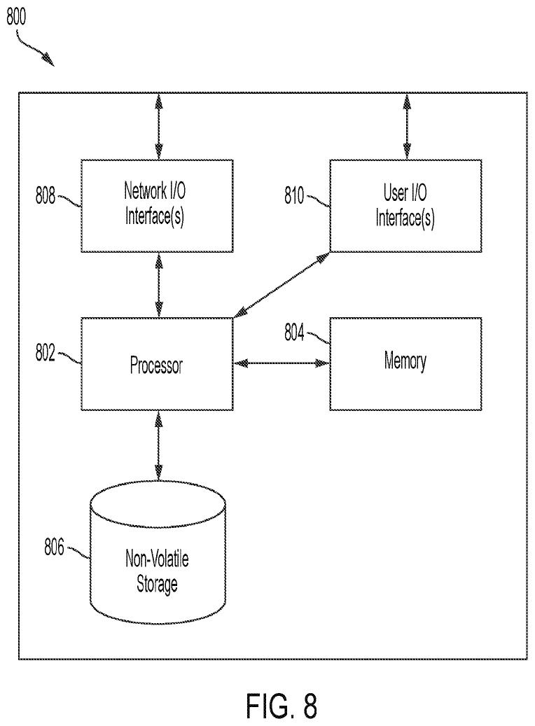

An illustrative implementation of a computer system 800 that may be used to perform any of the aspects of the techniques and embodiments disclosed herein is shown in FIG. 8. For example, the computer system 800 may be configured to implement the neural network 100 of FIG. 1A. The computer system 800 may be configured to train and execute the neural networks as described in FIGS. 1-5. The computer system 800 may include one or more processors 802 and one or more non-transitory computer-readable storage media (e.g., memory 804 and one or more non-volatile storage media 806) and a display 810. The one or more processors 802 may control writing data to and reading data from the memory 804 and the non-volatile storage device 806 in any suitable manner, as the aspects of the invention described herein are not limited in this respect. To perform functionality and/or techniques described herein, the processor(s) 802 may execute one or more instructions stored in one or more computer-readable storage media (e.g., the memory 804, storage media, etc.), which may serve as non-transitory computer-readable storage media storing instructions for execution by the processor 802.

In connection with techniques described herein, code used to, for example, train and/or run the neural network described in the present disclosure may be stored on one or more computer-readable storage media of computer system 800. Processor(s) 802 may execute any such code to provide any techniques for detecting anomalies as described herein. Any other software, programs or instructions described herein may also be stored and executed by computer system 800. It will be appreciated that computer code may be applied to any aspects of methods and techniques described herein. For example, computer code may be applied to interact with an operating system to detect anomalies through conventional operating system processes.

The various methods or processes outlined herein may be coded as software that is executable on one or more processors that employ any one of a variety of operating systems or platforms. Additionally, such software may be written using any of numerous suitable programming languages and/or programming or scripting tools, and also may be compiled as executable machine language code or intermediate code that is executed on a virtual machine or a cloud or other framework via a network interface 808.

In this respect, various inventive concepts may be embodied as at least one non-transitory computer readable storage medium (e.g., a computer memory, one or more floppy discs, compact discs, optical discs, magnetic tapes, flash memories, circuit configurations in Field Programmable Gate Arrays or other semiconductor devices, etc.) encoded with one or more programs that, when executed on one or more computers or other processors, implement the various embodiments of the present invention. The non-transitory computer-readable medium or media may be transportable, such that the program or programs stored thereon may be loaded onto any computer resource to implement various aspects of the present invention as discussed above.

The terms “program,” “software,” and/or “application” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present invention need not reside on a single computer or processor, but may be distributed in a modular fashion among different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in non-transitory computer-readable storage media in any suitable form. Data structures may have fields that are related through location in the data structure. Such relationships may likewise be achieved by assigning storage for the fields with locations in a non-transitory computer-readable medium that convey relationship between the fields. However, any suitable mechanism may be used to establish relationships among information in fields of a data structure, including through the use of pointers, tags or other mechanisms that establish relationships among data elements.

Various inventive concepts may be embodied as one or more methods, of which examples have been provided. The acts performed as part of a method may be ordered in any suitable way. Accordingly, embodiments may be constructed in which acts are performed in an order different than illustrated, which may include performing some acts simultaneously, even though shown as sequential acts in illustrative embodiments.

Some embodiments are directed to a system for predicting properties of a molecule, the system comprising: at least one processor configured to provide an input molecule to a neural network model and use the neural network model to predict one or more properties of the input molecule, wherein the neural network comprises: (1) an atom embedding layer configured to convert atom features of the input molecule to an atom representation; (2) a bond embedding layer configured to convert bond features of the input molecule to a bond representation; (3) a graph neural network comprising at least one layer, the at least one layer configured to update the atom representation base at least in part on the bond representation; (4) a molecule embedding layer configured to generate a molecule representation based on the updated atom representation; and (5) a target layer configured to predict one or more properties of the molecule based on the molecule representation.

In some embodiments, the target layer comprises: a first layer configured to predict a value for at least one property based on the molecule representation; and a second layer configured to predict a deviation value for the at least one property based on the molecule representation.

In some embodiments, the target layer comprises at least one layer configured to generate a plurality of values each indicating a likelihood of the input molecule belonging to a corresponding class of a plurality of classes.

In some embodiments, the atom features of the input molecule comprise one or more features of the input molecule comprising: for each atom in the input molecule, an atom number, a chirality, a formal charge, and/or a hydrogen count.

In some embodiments, the bond features of the input molecule comprise one or more features of the input molecule comprising: for each bonding of atoms in the input molecule, a bond type, a bond direction, and/or a shortest path distance.

In some embodiments, the atom embedding layer is configured to perform a linear transformation over the atom features followed by an activation function to generate the atom representation.

In some embodiments, the atom embedding layer is further configured to normalize the atom features before performing the linear transformation.

In some embodiments, the bond embedding layer is configured to perform a linear transformation over the bond features followed by an activation function to generate the bond representation.

In some embodiments, the bond embedding layer is further configured to normalize the bond features before performing the linear transformation.

In some embodiments, the molecule embedding layer is configured to perform one or more linear transformations over the updated atom representation, each linear transformation followed by an activation function, to generate the molecule representation.

In some embodiments, the graph neural network comprises a graph transformer network, where the neural network includes an atom final layer and a summation operation coupled between the graph transformer network and the molecular layer, wherein: (1) the atom final layer is configured to perform a linear transformation over the updated atom representation to pool the updated atom representation; and (2) the summation operation is configured to sum over the pooled updated atom representation along an atom axis.

In some embodiments, the at least one layer is a graph transformer layer comprising a first residual connection configured to update the bond representation with the atom representation.

In some embodiments, the at least one layer further comprises an attention network coupled to the first residual connection and configured to use the atom representation and the updated bond representation from the first residual connection to generate output.

In some embodiments, the attention network comprises: an attention layer configured to generate one or more attention scores based on the updated bond representation; a message layer configured to generate one or more bond messages based on the updated bond representation; wherein the attention network is configured to use the atom representation and a combination of one or more attention scores and the one or more bond messages to generate the output, wherein the combination comprises element-wise multiplication values calculated from the one or more attention scores and the one or more bond messages.

In some embodiments, the attention network further comprises an atom forward-feed neural network coupled to the attention network and configured to generate the updated atom representation based on the output from the attention network.

In some embodiments, the at least one layer further comprises a second residual connection coupled to the attention network and configured to update the atom representation with the output of the attention network atom representation to generate the updated atom representation.

In some embodiments, the graph neural network is a graph convolutional neural network and further configured to update the bond representation based at least in part on the atom representation.

In some embodiments, the neural network further comprises: an atom final layer configured to perform a linear transformation over the updated atom representation to pool the updated atom representation; a bond final layer configured to perform a linear transformation over the updated bond representation to pool the updated bond representation; and a summation operation coupled between the atom final layer, the bond final layer and the molecular layer. The summation operation is configured to sum over the pooled updated atom representation and the pooled updated bond representation to generate a molecule representation according to which molecule each atom in the atom representation belongs.

In some embodiments, the GCNN comprises at least one GCNN layer comprising: an atom message projection configured to project the atom representation to generate an intermediate atom representation; and a first GCNN summation configured to add the intermediate atom representation to the bond representation to generate the updated bond representation.

In some embodiments, the at least one GCNN layer further comprises: a bond message projection configured to project the updated bond representation to generate an intermediate bond representation; and a second GCNN summation configured to update the atom representation to generate the updated atom representation based on the intermediate bond representation.

Some embodiments are directed to method for predicting properties of a molecule, the method comprising, using at least one processor, using a neural network model to predict one or more properties of an input molecule, by: (1) converting atom features of the input molecule to an atom representation; (2) converting bond features of the input molecule to a bond representation; (3) using a graph neural network comprising at least one layer to update the atom representation base at least in part on the bond representation; (4) generating a molecule representation based on the updated atom representation; and (5) predicting one or more properties of the molecule based on the molecule representation.

In some embodiments, predicting the one or more properties of the molecule comprises: predicting a value for at least one property based on the molecule representation; and predicting a deviation value for the at least one property based on the molecule representation.

In some embodiments, predicting the one or more properties of the molecule comprises generating a plurality of values each indicating a likelihood of the input molecule belonging to a corresponding class of a plurality of classes.

In some embodiments, the atom features of the input molecule comprise one or more features of the input molecule comprising: for each atom in the input molecule, an atom number, a chirality, a formal charge, and/or a hydrogen count.

In some embodiments, the bond features of the input molecule comprise one or more features of the input molecule comprising: for each bonding of atoms in the input molecule, a bond type, a bond direction, and/or a shortest path distance.

In some embodiments, converting the atom features of the input molecule to the atom representation comprises: performing a linear transformation over the atom features followed by an activation function to generate the atom representation.

In some embodiments, converting the atom features of the input molecule to the atom representation further comprises normalizing the atom features before performing the linear transformation.

In some embodiments, converting the bond features of the input molecule to the bond representation comprises performing a linear transformation over the bond features followed by an activation function to generate the bond representation.

In some embodiments, converting the bond features of the input molecule to the bond representation further comprises normalizing the bond features before performing the linear transformation.

In some embodiments, generating the molecule representation based on the updated atom representation comprises performing one or more linear transformations over the updated atom representation, each linear transformation followed by an activation function, to generate the molecule representation.

In some embodiments, the graph neural network comprises a graph transformer network. The method further comprises: performing a linear transformation over the updated atom representation to pool the updated atom representation; and summing over the pooled updated atom representation along an atom axis.

In some embodiments, using the graph transformer network comprises using a first residual connection of the at least one layer of the graph transformer network to update the bond representation with the atom representation.

In some embodiments, using the graph transformer network further comprises using an attention network coupled to the first residual connection of the at least one layer to use the atom representation and the updated bond representation from the first residual connection to generate output.

In some embodiments, using the attention network comprises: by an attention layer of the attention network, generating one or more attention scores based on the updated bond representation; by a message layer of the attention network, generating one or more bond messages based on the updated bond representation; and using the atom representation and a combination of one or more attention scores and the one or more bond messages to generate the output, wherein the combination comprises element-wise multiplication values calculated from the one or more attention scores and the one or more bond messages.

In some embodiments, the method further comprises using an atom forward-feed neural network to generate the updated atom representation based on the output from the attention network.

In some embodiments, using the graph transformer network further comprises using a second residual connection coupled to the attention network the at least one graph transformer layer to update the atom representation with the output of the attention network atom representation to generate the updated atom representation.

In some embodiments, the graph neural network is a graph convolutional neural network. The method further comprises updating the bond representation based at least in part on the atom representation.

In some embodiments, the method further comprises: using an atom final layer to perform a linear transformation over the updated atom representation to pool the updated atom representation; using a bond final layer to perform a linear transformation over the updated bond representation to pool the updated bond representation; and using a summation operation to sum over the pooled updated atom representation and the pooled updated bond representation to generate a molecule representation according to which molecule each atom in the atom representation belongs.

In some embodiments, updating the bond representation comprises, by at least one GCNN layer: using an atom message projection to project the atom representation to generate an intermediate atom representation; and using a first GCNN summation to add the intermediate atom representation to the bond representation to generate the updated bond representation.

In some embodiments, updating the atom representation comprises, by the at least one GCNN layer: using a bond message projection to project the updated bond representation to generate an intermediate bond representation; and using a second GCNN summation to update the atom representation to generate the updated atom representation based on the intermediate bond representation.

Some embodiments are directed to a non-transitory computer-readable media comprising instructions that, when executed, cause at least one processor to perform operations comprising: using a neural network model to predict one or more properties of an input molecule, by: (1) converting atom features of the input molecule to an atom representation; (2) converting bond features of the input molecule to a bond representation; (3) using a graph neural network comprising at least one layer to update the atom representation base at least in part on the bond representation; (4) generating a molecule representation based on the updated atom representation; and (5) predicting one or more properties of the molecule based on the molecule representation.

In some embodiments, predicting the one or more properties of the molecule comprises: predicting a value for at least one property based on the molecule representation; and predicting a deviation value for the at least one property based on the molecule representation.

In some embodiments, predicting the one or more properties of the molecule comprises: generating a plurality of values each indicating a likelihood of the input molecule belonging to a corresponding class of a plurality of classes.

In some embodiments, the atom features of the input molecule comprise one or more features of the input molecule comprising: for each atom in the input molecule, an atom number, a chirality, a formal charge, and/or a hydrogen count.

U.S. Pat. Apl. No. 63/293,608 is incorporated herein by reference in its entirety.

In some embodiments, the bond features of the input molecule comprise one or more features of the input molecule comprising: for each bonding of atoms in the input molecule, a bond type, a bond direction, and/or a shortest path distance.

The indefinite articles “a” and “an,” as used herein in the specification and in the claims, unless clearly indicated to the contrary, should be understood to mean “at least one.” As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This allows elements to optionally be present other than the elements specifically identified within the list of elements to which the phrase “at least one” refers, whether related or unrelated to those elements specifically identified.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting.

Various aspects are described in this disclosure, which include, but are not limited to, the following aspects:

1. A system for predicting properties of a molecule, the system comprising:
at least one processor configured to provide an input molecule to a neural network model and use the neural network model to predict one or more properties of the input molecule, wherein the neural network comprises:
an atom embedding layer configured to convert atom features of the input molecule to an atom representation;
a bond embedding layer configured to convert bond features of the input molecule to a bond representation;
a graph neural network comprising at least one layer, the at least one layer configured to update the atom representation base at least in part on the bond representation;
a molecule embedding layer configured to generate a molecule representation based on the updated atom representation; and
a target layer configured to predict one or more properties of the molecule based on the molecule representation.

2. The system of claim 1, wherein the target layer comprises:
a first layer configured to predict a value for at least one property based on the molecule representation; and
a second layer configured to predict a deviation value for the at least one property based on the molecule representation.

3. The system of claim 1, wherein the target layer comprises at least one layer configured to generate a plurality of values each indicating a likelihood of the input molecule belonging to a corresponding class of a plurality of classes.

4. The system of claim 1, wherein the atom features of the input molecule comprise one or more features of the input molecule comprising: for each atom in the input molecule, an atom number, a chirality, a formal charge, and/or a hydrogen count.

5. The system of claim 1, wherein the bond features of the input molecule comprise one or more features of the input molecule comprising: for each bonding of atoms in the input molecule, a bond type, a bond direction, and/or a shortest path distance.

6. The system of claim 1, wherein the atom embedding layer is configured to perform a linear transformation over the atom features followed by an activation function to generate the atom representation.

7. (canceled)

8. The system of claim 1, wherein the bond embedding layer is configured to perform a linear transformation over the bond features followed by an activation function to generate the bond representation.

9. (canceled)

10. The system of claim 1, wherein the molecule embedding layer is configured to perform one or more linear transformations over the updated atom representation, each linear transformation followed by an activation function, to generate the molecule representation.

11. The system of claim 1, wherein the graph neural network comprises a graph transformer network, and wherein the neural network further comprises an atom final layer and a summation operation coupled between the graph transformer network and the molecular layer, wherein:
the atom final layer is configured to perform a linear transformation over the updated atom representation to pool the updated atom representation; and
the summation operation is configured to sum over the pooled updated atom representation along an atom axis.

12. The system of claim 11, wherein the at least one layer is a graph transformer layer comprising a first residual connection configured to update the bond representation with the atom representation.

13. The system of claim 12, wherein the at least one layer further comprises an attention network coupled to the first residual connection and configured to use the atom representation and the updated bond representation from the first residual connection to generate output.

14. The system of claim 13, wherein the attention network comprises:
an attention layer configured to generate one or more attention scores based on the updated bond representation;
a message layer configured to generate one or more bond messages based on the updated bond representation;
wherein the attention network is configured to use the atom representation and a combination of one or more attention scores and the one or more bond messages to generate the output, wherein the combination comprises element-wise multiplication values calculated from the one or more attention scores and the one or more bond messages.

15. The system of claim 14, wherein the attention network further comprises an atom forward-feed neural network coupled to the attention network and configured to generate the updated atom representation based on the output from the attention network.

16. The system of claim 13, wherein the at least one layer further comprises a second residual connection coupled to the attention network and configured to update the atom representation with the output of the attention network atom representation to generate the updated atom representation.

17. The system of claim 1, wherein the graph neural network is a graph convolutional neural network (GCNN) and further configured to update the bond representation based at least in part on the atom representation.

18. The system of claim 17, wherein the neural network further comprises:
an atom final layer configured to perform a linear transformation over the updated atom representation to pool the updated atom representation;
a bond final layer configured to perform a linear transformation over the updated bond representation to pool the updated bond representation; and
a summation operation coupled between the atom final layer, the bond final layer and the molecular layer, the summation operation configured to sum over the pooled updated atom representation and the pooled updated bond representation to generate a molecule representation according to which molecule each atom in the atom representation belongs.

19. The system of claim 17, wherein the GCNN comprises at least one GCNN layer comprising:
an atom message projection configured to project the atom representation to generate an intermediate atom representation; and
a first GCNN summation configured to add the intermediate atom representation to the bond representation to generate the updated bond representation.

20. The system of claim 19, wherein the at least one GCNN layer further comprises:
a bond message projection configured to project the updated bond representation to generate an intermediate bond representation; and
a second GCNN summation configured to update the atom representation to generate the updated atom representation based on the intermediate bond representation.

21. A method for predicting properties of a molecule, the method comprising, using at least one processor:
using a neural network model to predict one or more properties of an input molecule, by:
converting atom features of the input molecule to an atom representation;
converting bond features of the input molecule to a bond representation;
using a graph neural network comprising at least one layer to update the atom representation base at least in part on the bond representation;
generating a molecule representation based on the updated atom representation; and
predicting one or more properties of the molecule based on the molecule representation.

22-40. (canceled)

41. A non-transitory computer-readable media comprising instructions that, when executed, cause at least one processor to perform operations comprising:
using a neural network model to predict one or more properties of an input molecule, by:
converting atom features of the input molecule to an atom representation;
converting bond features of the input molecule to a bond representation;
using a graph neural network comprising at least one layer to update the atom representation base at least in part on the bond representation;
generating a molecule representation based on the updated atom representation; and
predicting one or more properties of the molecule based on the molecule representation.

42-45. (canceled)
